(19) United States
(12) Patent Application Publication
(10) Pub. No.: US 2024/0020718 A1
(43) Pub. Date: Jan. 18, 2024

(54) ACTIVITY RECRUITMENT PLATFORM

(71) Applicant: Oklahoma Blood institute, Oklahoma City, OK (US)

(72) Inventors: Justin Ryan Redwine, Cushing, OK (US); John Brooks Armitage, Oklahoma City, OK (US)

(21) Appl. No.: 18/352,102

(22) Filed: Jul. 13, 2023

Related U.S. Application Data
(60) Provisional application No. 63/368,405, filed on Jul. 14, 2022.

(51) Int. Cl. G06Q 30/0214; G16H 40/20

(52) U.S. Cl. CPC G06Q 30/0214; G16H 40/20

(57) ABSTRACT

A system including a processor and computer readable medium (CRM) is described. The CRM stores instructions causing the processor to: display a user interface to enable a campaign activity (CA) on a user system. The CA is associated with a campaign and has a campaign activity property, and the user interface includes input fields corresponding to recruit information associated with a particular recruit; receive a user ID, the recruit information, and an activity indicator identifying the CA; schedule the CA identified by the activity indicator with the recruit information; receive a recruit identifier identifying the particular recruit and a recruit status indicative of whether the recruit completed the campaign activity; and allocate, to the user of the user system, a donation or a monetary value based upon the campaign activity property when the recruit status indicates the particular recruit successfully completed the CA.

### CROSS-REFERENCE TO RELATED APPLICATIONS

The present application is a non-provisional application claiming priority to U.S. Provisional Application No. 63/368,405 filed Jul. 14, 2022, the entire content of which is incorporated herein by reference in its entirety.

### BACKGROUND OF THE INVENTION

Donations are central to the operation of many non-profit organizations. Whether the donation is of money, a product, or a service, there is often a conversion gap between what a potential donor offers to donate and what the potential donor actually donates.

Soliciting or recruiting donations can be time consuming and costly. The organization has to make sure it has staffed enough recruiters to find potential donors. Additionally, the organization has to maintain the staff and the materials to help find and recruit potential donors. Additionally, the organization may plan resources around prospective donations. In instances when actual donations fall short of prospective donations, the organization may inadvertently exhaust resources without obtaining an actual donation.

For example, if the organization is a blood donation center, the organization may schedule donors over a period of time during which the organization ensures that staff are present to assist with the blood donation and that donors are not required to wait for extended periods of time to donate blood. When a donor is scheduled to donate blood, but fails to show up, not only does the organization lose out on the donation from the scheduled donor, but also loses out on a donation from another donor that could have used that time slot and still has to provide staff for that time slot even when no one is donating. The more donors that are scheduled and do not show up, i.e., the greater the conversion gap, the more costly obtaining each donation that is received becomes.

Donors have been scheduled through the use of call centers hired by the blood donation centers. Further, blood donation centers have used SMS messages, MMS messages, emails, and direct mails in an attempt to reach out and encourage donors to donate.

Thus, there is a need for a donor recruitment platform that is capable of shrinking the conversion gap, i.e., increasing the number of completed donations.

### SUMMARY OF THE INVENTION

As discussed above, there are various ways to schedule donors for a donation activity. But potential donors do not want to be bothered by an unsolicited telephone call, or respond to an unsolicited SMS message. Rather, a potential donor that receives a request from someone they know and trust is more likely to donate. With this in mind, the presently disclosed inventive concepts are a technological solution to the problem of scheduling donors for a donation activity.

In some embodiments, the presently disclosed inventive concepts includes a system, comprising a processor and a memory. The memory comprises one or more non-transitory processor-readable medium storing processor-executable instructions that when executed by the processor cause the processor to: display, on a user system of a user, a user interface to enable a campaign activity associated with a campaign, where the user interface has one or more campaign activity property and one or more input fields corresponding to recruit information; receive, from the user system, a user ID, the recruit information from the one or more input fields, and an activity indicator identifying a particular one of the one or more campaign activity; schedule the particular one of the one or more campaign activity with the recruit information; receive, from a campaign system, a recruit identifier and a recruit status, the recruit identifier identifying the particular recruit and the recruit status indicative of whether the recruit completed the campaign activity; and allocate, to the user of the user system, at least one of a donation or a monetary value based upon the one or more campaign activity property when the recruit status is indicative of the recruit successfully completing the campaign activity. The recruit information corresponds to a particular recruit and the campaign activity is a tissue donation.

### BRIEF DESCRIPTION OF THE DRAWINGS

These and other objects and features of the present invention will be more fully disclosed or rendered obvious by the following detailed description of the invention, which is to be considered together with the accompanying drawings wherein like numbers refer to like parts, and further wherein:

FIG. 1 illustrates a block diagram of an exemplary recruitment platform in accordance with the present disclosure;

FIG. 2 illustrates a block diagram of another exemplary recruitment platform in accordance with the present disclosure;

FIG. 3A-3F illustrate screenshots of an exemplary embodiment of an associated log-in and registration of a recruitment platform in accordance with the present disclosure;

FIG. 4A-4C illustrate screenshots of an exemplary recruit registration of a recruitment platform in accordance with the present disclosure; and

FIG. 5 illustrates a screenshot of an exemplary embodiment of a notification screen of a recruitment platform in accordance with the present disclosure.

### DETAILED DESCRIPTION

Before explaining at least one embodiment of the presently disclosed and claimed inventive concepts in detail, it is to be understood that the presently disclosed and claimed inventive concepts are not limited in their application to the details of construction, experiments, exemplary data, and/or the arrangement of the components set forth in the following description or illustrated in the drawings. The presently disclosed and claimed inventive concepts are capable of other embodiments or of being practiced or carried out in various ways. Also, it is to be understood that the phraseology and terminology employed herein is for purpose of description and should not be regarded as limiting.

In the following detailed description of embodiments of the inventive concepts, numerous specific details are set forth in order to provide a more thorough understanding of the inventive concepts. However, it will be apparent to one of ordinary skill in the art that the inventive concepts within the disclosure may be practiced without these specific details. In other instances, certain well-known features may not be described in detail in order to avoid unnecessarily complicating the instant disclosure.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherently present therein.

Unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

The term “and combinations thereof” as used herein refers to all permutations or combinations of the listed items preceding the term. For example, “A, B, C, and combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. A person of ordinary skill in the art will understand that typically there is no limit on the number of items or terms in any combination, unless otherwise apparent from the context.

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

The use of the terms “at least one” and “one or more” will be understood to include one as well as any quantity more than one, including but not limited to each of, 2, 3, 4, 5, 10, 15, 20, 30, 40, 50, 100, and all integers and fractions, if applicable, therebetween. The terms “at least one” and “one or more” may extend up to 100 or 1000 or more, depending on the term to which it is attached; in addition, the quantities of 100/1000 are not to be considered limiting, as higher limits may also produce satisfactory results.

Further, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein qualifiers such as “about,” “approximately,” and “substantially” are intended to signify that the item being qualified is not limited to the exact value specified, but includes some slight variations or deviations therefrom, caused by measuring error, manufacturing tolerances, stress exerted on various parts, wear and tear, and combinations thereof, for example.

Software may include one or more computer readable instructions that when executed by one or more components cause the component to perform a specified function. It should be understood that algorithms or process instructions described herein may be stored on one or more non-transitory computer readable medium. Exemplary non-transitory computer readable medium may include random access memory, read only memory, flash memory, and/or the like. Such non-transitory computer readable mediums may be electrically based, optically based, and/or the like.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), field programmable gate array (FPGA), a combination of hardware and software, and/or the like. The term “processor” as used herein means a single processor or multiple processors working independently or together to collectively perform a task.

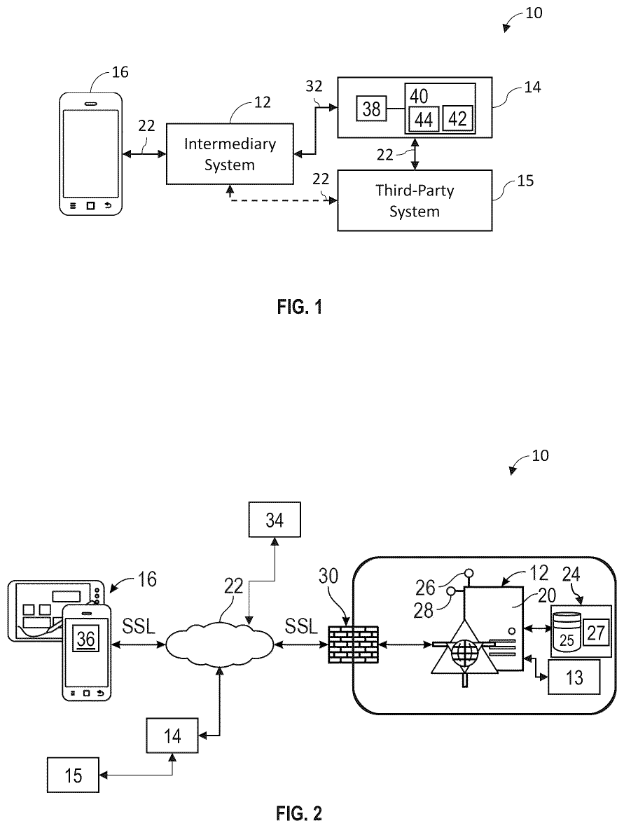

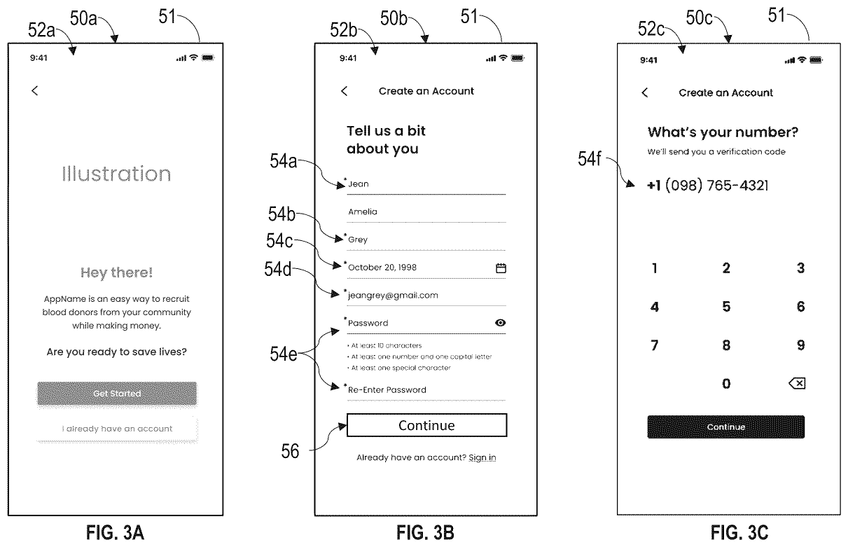

Referring now to the Figures, and in particular to FIG. 1, shown therein is a block diagram of an exemplary embodiment of a recruitment platform 10 in accordance with the present disclosure. Generally, the recruitment platform 10 may be configured to provide a user with an activity recruit (“recruit”) scheduling platform to earn monetary donations (e.g., micro-donations) and/or monetary funds when the recruit completes an activity, such as a donation or a non-donation. Such opportunities may be provided by a campaign host, e.g., a non-profit organization such as a blood center. The user may be provided access to one or more campaign for recruits, and upon the completion of a campaign activity (e.g., a blood donation) by a recruit, the user who recruited the recruit and/or scheduled the campaign activity for the recruit may earn a pre-determined amount of funds. The recruitment platform 10 may provide the campaigns to the user, and upon completion of the campaign activity by the recruit, determine an allocation of funds to be provided to the user who recruited the recruit. The recruitment platform 10 may include one or more intermediary system 12 (hereafter intermediary system 12), one or more accounting system 13 (hereafter accounting system 13) (see FIG. 2), one or more campaign system 14 (hereafter campaign system 14), one or more third-party system 15 (hereafter third-party system 15), and one or more user system 16 (hereafter user system 16). When the recruitment platform 10 includes multiple user systems 16, at least some of the user systems 16 are associated with and/or used by particular users.

In one non-limiting example, one or more non-profit organization may solicit one or more campaign for inclusion in the recruitment platform 10. In some embodiments, the recruitment platform 10 may be controlled by the campaign host, e.g., one or more non-profit organizations. In some embodiments, the recruitment platform 10 may be controlled by one or more other organization. Generally, the non-profit organization (or other organization) may create one or more campaigns. The one or more campaigns may be directed to attracting recruits to complete one or more campaign activity such as a donation for the non-profit organization. A user may access the user system 16 to view the one or more campaigns and elect to participate in attracting recruits to complete the one or more campaign activities. The user may then use the user system 16 to schedule one or more recruit to complete the one or more campaign activity. Upon completion of the campaign activity by a recruit, a campaign activity amount may be assigned to the user. In one embodiment, the one or more campaign activity may be a tissue donation activity, such as one or more of a plasma donation activity, a platelet donation activity, and a blood donation activity. As used herein, a “tissue donation” may refer to a donation of a biological tissue and/or a biological tissue component/constituent.

In one embodiment, the accounting system 13 may account for and/or track each campaign activity amount assigned to each user. The accounting system 13 may be a system, or systems, that is able to embody and/or execute the logic of the processes described herein. Logic embodied in the form of software instructions and/or firmware may be executed on appropriate hardware. For example, logic embodied in the form of software instructions or firmware may be executed on a computer system, a distributed processing computer system, and/or the like.

The intermediary system 12 may provide the one or more campaign to users via a network 22 to the user system 16.

In some embodiments, the intermediary system 12 and/or the accounting system 13 may account for and/or obtain funds from one or more campaign system 14 (e.g., a campaign host) based on completion of one or more campaign activity by a recruit for the user to earn monetary donations and/or monetary values/funds and distribute at least a portion of the funds to a user account for the benefit of the user. In some embodiments, collection of funds from the one or more campaign system 14 may be automatic (i.e., without human intervention by using an algorithm that uses predetermined inputs to effect steps within the process) and based on accounting provided by the intermediary system 12 and/or the accounting system 13 of the recruits' completion of the one or more campaign activities.

Alternatively, in some embodiments, the accounting system 13 may be configured to provide an accounting of the one or more campaign activities and provide the accounting to the campaign system 14. In this example, the campaign system 14 may distribute funds to the user account directly via any predetermined methodology, such as electronic transfer, wiring and the like, or the campaign system 14 may send one or more communication to a third-party payment system 34 as described below.

In some embodiments, the intermediary system 12 may provide the accounting of the one or more activities and associated users to the campaign system 14 for review. Upon review and acceptance of the accounting, the campaign system 14 may transmit funds to the intermediary system 12 for distribution to pre-identified ones of the user accounts. In some embodiments, distribution of funds from the one or more campaign system 14 may be automatic (i.e., without human intervention by using an algorithm that uses predetermined inputs to effect steps within the process) and based on accounting provided by the accounting system 13 and pre-determined allocation guidance for distribution of funds.

In some embodiments, the intermediary systems 12 may be configured to provide the user system 16 the one or more campaigns so that the user can earn monetary donations and/or monetary funds. For example, the intermediary system 12 may transmit the one or more campaigns from the campaign system 14 to the user system 16 such that the user can access the one or more campaigns. A “campaign” as used herein is a sequence of instructions and data associated with at least one particular campaign activity that identifies the particular campaign activity(ies) and enables scheduling a donor to participate in the campaign activity(ies). Upon completion of a campaign activity by a recruit, the accounting system 13 may account for and/or collect funds from the campaign system 14 based on completion of the campaign activity, and distribute at least a portion of the funds to the user account or cause the third-party payment system 34 to distribute at least a portion of the funds to the user account.

In one embodiment, for example, the campaign activity may be a blood drive or a blood donation whereby a non-profit organization is soliciting blood donors. In this example, users may schedule (via the user system 16) one or more recruit (e.g., blood donor) to complete one or more campaign activity (e.g., blood donation activity, plasma donation activity, etc.) of a particular campaign. When the recruit completes the campaign activity (e.g., when the recruit donates blood, plasma, etc.), the intermediary system 12 and/or the accounting system 13 may allocate campaign activity funds to the user's user account.

In one embodiment, based on properties of the campaign in the campaign system 14, the campaign activity funds may be a predetermined monetary amount based on the particular campaign activity completed. For example, if a campaign includes a first campaign activity and a second campaign activity, the first campaign activity may have a first campaign activity fund and the second campaign activity may have a second campaign activity fund. In some embodiments, the first campaign activity fund and the second campaign activity fund are different, however, in other embodiments, the first campaign activity fund and the second campaign activity fund are the same.

In some embodiments, the campaign activity fund may be dependent on the recruit's attempt to complete the campaign activity. For example, in the campaign to recruit blood donors, the recruit may arrive at a blood donation location, but be unable to complete the process through no fault of their own. In this case, the campaign activity fund allocated to the user that scheduled the recruit may be lesser than the campaign activity fund allocated to the user that scheduled the recruit had the recruit been able to successfully complete the campaign activity. On other embodiments, however, the campaign activity funds may be awarded in full any time a recruit attempts to complete the campaign activity. How campaign activity funds are calculated and distributed may be determined when the campaign is established in the campaign system 14.

The intermediary system 12 may be a system or systems that are able to embody and/or execute the logic of the processes described herein. Logic embodied in the form of software instructions and/or firmware may be executed on appropriate hardware. For example, logic embodied in the form of software instructions or firmware may be executed on a computer system, a distributed processing computer system, and/or the like. In some embodiments, the logic may be implemented in a stand-alone environment operating on a single computer system, and/or logic may be implemented in a networked environment, such as a distributed system using multiple computers and/or processors. To that end, one or more elements of logic embodied in the form of software instructions or firmware may be executed in a stand-alone environment and one or more elements of the logic may be executed in the distributed system.

Referring to FIGS. 1 and 2, in some embodiments, the intermediary system 12 may include one or more processor 20 configured to communicate with the one or more user system 16 over a network 22. The one or more processor 20 may work together and/or independently to execute processor executable code. Additionally, the intermediary system 12 may include one or more memory 24 storing processor executable code and one or more database 25. In some embodiments, each element of the intermediary system 12 may be partially or completely network-based or cloud-based, and may or may not be located in a single physical location.

In one embodiment, the intermediary system 12 may incorporate the accounting system 13. In other embodiments, the accounting system 13 may be executed on the processor 20, store one or more data on the memory 24, and communicate with the intermediary system 12

The one or more processor 20 may be implemented as a single or plurality of processors working together, or independently, to execute the logic as described herein. Exemplary embodiments of the one or more processor 20 may include, but are not limited to, a digital signal processor (DSP), a central processing unit (CPU), a field programmable gate array (FPGA), a microprocessor, a multi-core processor, and/or combinations thereof, for example. The one or more processor 20 may be capable of communicating via the network 22 or a separate network (e.g., analog, digital, optical, and/or the like) via one or more ports (e.g., physical or virtual ports) using a network protocol. The one or more processor 20 may be capable of reading and/or executing processor executable code and/or capable of creating, manipulating, retrieving, altering, and/or storing data structure into one or more memory (e.g., the one or more memory 24).

In some embodiments, the one or more memory 24 is a non-transitory processor-readable medium storing processor-executable instructions and may be located in the same physical location as the one or more processor 20. Alternatively, the one or more memory 24 may be implemented as a “cloud memory” (e.g., the one or more memory 24 may be partially or completely based on or accessed using a network, such as the network 22). In one embodiment, the one or more memory 24 may be implemented as a first memory (e.g., a local memory) and a second memory. For example, the first memory may be a local memory storing the database 25 and the software application 27 and the second memory may store one or more multimedia file or webpage, such as a video file, an HTML file, a PHP file, an audio file, a document file, an image file, and the like. In one embodiment, the second memory may store images, PDFs, videos, and other documentation such as onboarding documentation.

The one or more memory 24 may store processor executable code and/or information comprising one or more databases 25 and program logic, such as a software application 27. For example, the database 25 hosted by the intermediary system 12 may store data indicative of an inventory of users accessing the user system 16, campaigns the users have joined, recruits the user(s) have scheduled and the schedule information, campaign activities the user's recruits have completed, data indicative of an amount of funds allocated to the user, communications (e.g., emails, text messages, calls, etc.) to or from users and recruits, and/or the like. The database 25 may be a relational database or a non-relational database. Examples of such databases 25 comprise, DB2©, Microsoft® Access, Microsoft® SQL Server (e.g., on-premise, Azure, or both), Oracle®, mySQL, PostgreSQL, MongoDB, Apache Cassandra, InfluxDB, Prometheus, Redis, Elasticsearch, TimescaleDB, Firebase, and/or the like. It should be understood that these examples have been provided for the purposes of illustration only and should not be construed as limiting the presently disclosed inventive concepts. The database 25 can be centralized or distributed across multiple systems or servers.

In one embodiment, the communications (e.g., emails, text messages, calls, etc.) to or from users and recruits may be provided, managed, and/or stored by a third-party communications provider, such as Twilio (San Francisco, CA) or SendGrid (Denver, CO). In other embodiments, the communications to or from users and recruits may be provided, managed, and/or stored by the intermediary system 12.

The intermediary system 12 may communicate with one or more user system 16 via the network 22. The network 22 may be almost any type of network. In some embodiments, the network 22 may use network topographies and/or protocols including, but not limited to Ethernet, TCP/IP, UDP, circuit switched paths, and/or combinations thereof. For example, in some embodiments, the network 22 may be implemented as a wireless network, a cellular network, a Global System for Mobile Communications (GSM) network, a code division multiple access (CDMA) network, a 3G network, a 4G network, an LTE network, a 5G network, a satellite network, an optical network, combinations thereof, and/or the like. It is also conceivable that in the near future, embodiments of the present disclosure may use more advanced networking topologies and/or protocols. Additionally, the one or more processor 20 and/or one or more memory 24 may be capable of and configured to communicate with each other via the network 22 or additional networks.

The network 22 may be secured using any desired secured networking protocol, such as a gateway server, a firewall, data encryption, public or private key cryptography infrastructure, secure socket layer protocol, hypertext transfer protocol secure, a virtual private network, a tunnel, secure shell, any combinations thereof, and/or the like. In some embodiments, the recruitment platform 10 may include a network security system 30. The network security system 30 may monitor and/or control incoming and outgoing network data based on one or more predetermined security guidelines. Generally, the network security system 30 may provide a barrier between the user system 16 and the intermediary systems 12. Additionally, in some embodiments, the network security system 30 may provide a barrier between the user system 16 and the intermediary system 12, and/or campaign system 14. The network security system 30 may be a host-based system running on the intermediary system 12 or a network system running between two or more networks (e.g., the Internet and an internal network of the intermediary system 12). In some embodiments, the network security system 30 may monitor and/or control traffic between two or more networks.

In one embodiment, the network security system 30 may provide an application programming interface (API) operable to receive one or more communication via the network 22 and expose one or more function or method provided in the software application 27 of the intermediary system 12. The API may be a set of routines, protocols, functions, and/or methods for interacting with the intermediary system 12 exposed as an API endpoint. The API may express a software component in terms of the software component's operations, inputs, outputs, and underlying types.

In one embodiment, the API defines functionalities that are independent of respective implementations, which allows definitions and implementations to vary without compromising each other. For example, different versions of the software application 27 may provide the same API such that a communication engaging with the API (e.g., with the API endpoint) may expect the same functionality regardless of the version of the software application 27, or for more than one version of the software application 27. In addition to accessing the software application 27, the API may provide access to one or more database 25 or to computer hardware, such as the processor 20 and the memory 24, or other hardware such as PCI/PCIe cards, video cards, etc. Additionally, the API may assist otherwise distinct applications with sharing data, which can further integrate and enhance functionalities of the applications, such as the software application 27 and/or the third-party system 15.

In one embodiment, the API is in the form of a library that includes specifications for routines, data structures, object classes, and variables. In other embodiments, notably SOAP and REST services, the API is a specification of remote calls exposed to API consumers. The API specification can take many forms, including an International 7 Standard, such as POSIX, vendor documentation, such as the Microsoft Windows API, a high-performance Remote Procedure Call (such as gRPC), or the libraries of a programming language, e.g., Standard Template Library in C++ or Java API. In one embodiment, the API may access one or more database, service, authentication, and/or integration provider, such as, for example, Firebase.

In one embodiment, the API may use a message broker provider or message queueing system to manage one or more API call. For example, the API may utilize RabbitMQ (VMware, Inc., Palo Alto, CA), MQTT (Oasis MQTT 5 Specification), or gRPC.

In one embodiment, the intermediary system 12 may communicate with the one or more third-party system 15 via the network 22.

In some embodiments, the intermediary system 12 may include one or more input device 26 and one or more output device 28. The one or more input devices 26 may be capable of receiving information directly from a user, processor, and/or environment, and transmit such information to the one or more processor 20 and/or the network 22. The one or more input device 26 may include, but are not limited to, implementation as a keyboard, touchscreen, mouse, trackball, microphone, fingerprint reader, infrared port, cell phone, PDA, controller, network interface, speech recognition, gesture recognition, eye tracking, brain-computer interface, combinations thereof, and/or the like. In one embodiment, the one or more input device 26 is integrated into the user system 16.

The one or more output device 28 may be configured to output information in a form perceivable by a user and/or processor(s). In some embodiments, the one or more output device 28 may be configured to output information automatically (i.e., without human intervention). For example, in some embodiments, the one or more output device 28 may be configured to print or display at a pre-determined time interval an accounting of users, monetary donations, agencies, activities, and/or the like. The one or more output device 28 may include, but are not limited to, implementation as a computer monitor, a screen, a touchscreen, a speaker, a website, a television set, an augmented reality system, a smart phone, a PDA, a cell phone, a fax machine, a printer, a laptop computer, an optical head-mounted display (OHMD), combinations thereof, and/or the like. In one embodiment, the one or more output device 28 is integrated into the user system 16.

In some embodiments, the intermediary system 12 may communicate with the one or more user system 16, and/or the campaign system 14 using any communication protocol (e.g., SOAP, XML, JSON, REST). For example, the intermediary system 12 may communication using a JSON Rest service communication protocol. In some embodiments, the intermediary system 12 may serve as the intermediary between all systems (i.e., the user system 16, the campaign system 14, and any third-party system 15). As such, in these embodiments, all requests from or to each system 14, 15, 16, etc., will be directed through the intermediary system 12.

As discussed above, the recruitment platform 10 may include the campaign system 14. The intermediary system 12 may communicate with the campaign system 14 for collection of funds, accounting of funds, and/or to provide one or more campaigns to the user system 16 from which the user system 16 may select a particular campaign. Additionally, in some embodiments, the intermediary system 12 may communicate with the campaign system 14 to determine one or more available schedule slot available for a user to schedule a recruit for a campaign activity.

The intermediary system 12 may communicate and/or transmit data with the campaign system 14 via a network 32. In some embodiments, network 22 and network 32 may be the same network. The network 32 may be implemented as a wireless and/or wired network (e.g., a network configured to allow bi-directional exchange of data and/or signals), and may permit bi-directional communication of information and/or data between the intermediary system 12 and the campaign system 14. The network 32 may use a variety of network protocols to permit bi-directional interface and communication of data and/or information between the intermediary system 12 and the campaign system 14. The network 32 may be secured using any desired secured networking protocol, such as a gateway server, a firewall, data encryption, public or private key cryptography infrastructure, secure socket layer protocol, hypertext transfer protocol secure, a virtual private network, a tunnel, secure shell, any combinations thereof, and/or the like.

In one embodiment, the third-party system 15 stores and provides one or more campaign having at least one campaign activity directly to the user system 16 and/or to the user system 16 via the intermediary system 12. For example, the third-party system 15 may be a scheduling provider having a database of campaign activity availability (e.g., available dates, times, and locations for which the user may schedule the recruit for a particular campaign activity). The intermediary system 12 may communicate with the third-party system 15 to determine one or more available schedule slot 83a-n for a particular campaign and may transmit recruit information to the third-party system 15 to schedule the recruit for a selected available schedule slot 83, as discussed in more detail below.

In another example, the campaign system 14 may provide one or more campaign activity availability stored on the one or more memory 24 to the user system 16 directly and/or to the user system 16 via the intermediary system 12. For example, the intermediary system 12 may be configured to communicate with the campaign system 14 to access the campaign activity availability (e.g., available dates, times, and locations for which the user may schedule the recruit for a particular campaign activity) directly, or provide access to the campaign activity availability to the user system 16.

In some embodiments, the intermediary system 12 may transmit one or more communications to the campaign system 14 assigning a particular recruit to a particular campaign activity availability based on input received from the user accessing the user system 16.

In some embodiments, the intermediary system 12 may communicate the campaign activity availability scheduled, along with recruit information, to the third-party system 15, the campaign system 14, and/or to the user system 16.

In one embodiment, recruit information may be information from or about the recruit needed to schedule the recruit for a particular campaign activity at a particular date, time, and/or location. The required recruit information may be determined when the campaign is established. The recruit information may include one or more of, for example, a first name, a last name, a location (e.g., an address or a general area such as a city), a date of birth, a phone number, an email address, and/or the like. In some embodiments, the recruit information is retained by the intermediary system 12 but is anonymized as a recruit ID when transmitted to the third-party system 15. For example, the intermediary system 12 may include a database, e.g., in the memory 24, to store the recruit information. The intermediary system 12 and/or the database in the memory 24 may provide a recruit ID for a particular recruit. The recruit ID may be provided to the third-party system 15 and/or the campaign system 14 such that the third-party system 15 and/or the campaign system 14 does not store or receive any recruit information when reserving a scheduled campaign activity date, time, and location for a particular recruit. In other embodiments, the intermediary system 12 may communicate one or more recruit information to a third-party system 15, e.g., as required by the third-party system 15.

In some embodiments, the intermediary system 12 and/or the accounting system 13 may distribute campaign activity funds to the user account. For example, the intermediary system 12 may directly distribute monetary funds to the user. In another non-limiting example, a third-party payment system 34 (e.g., PayPal, CashApp, Venmo, Stripe, a bank, or other financial service) may be used to distribute funds to the user. The intermediary system 12 may automatically initiate interaction with the third-party payment system 34 to authorize distribution of particular amount of funds to the user. The amount of funds distributed to a user may be determined by, for example, the user, the amount of funds earned to the user, e.g., by scheduling recruits, a pre-determined funds threshold, e.g., any time earned funds reaches a pre-set limit, and/or the like.

In some embodiments, the intermediary system 12 does not store or transmit financial data to the third-party payment system 34, but instead may direct a user to an interface for the third-party payment system 34 such that all financial information is provided from the user system 16 to the third-party payment system 34 without passing through the intermediary system 12. The third-party payment system 34 may be in communication with the intermediary system 12 and/or the accounting system 13 to provide an accounting of fund distribution to the intermediary system 12 and/or the accounting system 13.

In another non-limiting example, the intermediary system 12 and/or the accounting system 13 may automatically provide a distribution in physical form (e.g., a printed check) for mailing to a physical address of the user. In some embodiments, the intermediary system 12 and/or the accounting system 13 may automatically generate and provide an accounting of distributions at a pre-determined interval (e.g., weekly distribution accounting).

In some embodiments, the intermediary system 12 may generate and provide a report to the campaign system 14. The report may include information related to the users, recruits, and/or campaign system 14 including, but not limited to recruit ID, recruit information, the recruit's scheduled campaign activity, user information, amount of awarded funds for a particular campaign activity, dates of use of the recruitment platform, the number of individuals contacted, the number of recruits scheduled, the conversion rate of scheduled recruits to the recruits having completed the campaign activity, and/or the like.

The user system 16 may be implemented as a smartphone, a tablet, a laptop computer, a personal computer, a desktop computer, a computer terminal, a computer workstation, an e-book reader, a wireless network-capable handheld device, a personal digital assistant, a kiosk, a gaming system, and/or the like. Similar to the intermediary system 12, the user system 16 may be provided with one or more processors, one or more non-transitory processor readable medium, an input device, and an output device. The processor, the one or more non-transitory processor readable medium, the input device, and the output device of the user system 16 may be implemented similarly to or the same as the processor 20, the one or more memory 24, the input device 26, and the output device 28 respectively. The user system 16 may be configured to interface with the network 22, via a wired or wireless interface.

The user system 16 may store processor executable instructions or a recruitment software application 36. For example, the user system 16 may include a web browser and/or a native software application running on the user system 16 and configured to communicate with the intermediary system 12 over the network 22. The recruitment software application 36 on the user system 16 may be configured for accessing a website and/or communicating information and/or data with the intermediary system 12 over the network 22.

In some embodiments, the user system 16 may include the recruitment software application 36 (e.g., specialized program downloaded onto the user system 16), and communicate with the intermediary system 12 via the network 22 through the recruitment software application 36. In some embodiments, the network 22 may be the Internet and/or other network. For example, if the network 22 is the Internet, a primary user interface of fundraising platform software may be delivered through a series of web pages.

The campaign system 14 may be implemented as a smartphone, a tablet, a laptop computer, a personal computer, a desktop computer, a computer terminal, a computer workstation, an e-book reader, a wireless network-capable handheld device, a personal digital assistant, a kiosk, a gaming system, and/or the like. Similar to the intermediary system 12, the campaign system 14 may be provided with one or more processor 38, one or more memory 40 comprising a non-transitory processor readable medium, an input device, and an output device. The processor 38, the memory 40, the input device, and the output device of the campaign system 14 may be implemented similarly to or the same as the processor 20, the one or more memory 24, the input device 26, and the output device 28, respectively. The campaign system 14 may be configured to interface with the network 32, via a wired or wireless interface.

The campaign system 14 may store processor executable instructions or a software application, such as a campaign application 42. For example, the campaign system 14 may include a web browser and/or a native software application running on the campaign system 14 and configured to communicate with the intermediary system 12 over the network 32. The campaign application 42 on the campaign system 14 may be configured for accessing a website and/or communicating information and/or data with the intermediary system 12 over the network 32, such as via the API. In one embodiment, the campaign system 14 may be “containerized”, that is, the campaign system 14 may implement the campaign application 42 as one or more container on the campaign system 14 and executed by the processor 38. In some embodiments, the one or more container may be managed by a container orchestration service, such as Docker, Docker Compose, and/or Kubernetes, or the like.

In some embodiments, the campaign system 14 may include an application program (e.g., a campaign application 42, shown in FIG. 1) (e.g., specialized program downloaded onto the campaign system 14), and communicate with the intermediary system 12 via the network 32, e.g., through the application programming interface (API). In some embodiments, the network 32 may be the Internet and/or other network. For example, if the network 32 is the Internet, a campaign interface of the recruitment platform software may be delivered through a series of web pages. The campaign interface may include one or more inputs to receive campaign information, receive campaign activity information, receive campaign activity funds properties, receive campaign bank account information, and/or the like.

In one embodiment, the campaign system 14 may be managed and/or under the control of the campaign host. The campaign application 42, executed on the campaign system 14 being managed by the campaign host, may have an application version operable to communicate via the API. In another embodiment, a first campaign system executing a first campaign application having a first application version may be managed by a first campaign host while a second campaign system executing a second campaign application having a second application version may be managed by a second campaign host. The first campaign application and the second campaign application may communicate with the intermediary system 12 via the API even though the first application version is different than the second application version. In one embodiment, the memory 40 of the campaign system 14 may further store one or more partner database. The one or more partner database may store, for example, one or more scheduled campaign activity having one or more campaign activity property such as a campaign activity ID identifying the corresponding campaign activity, and a campaign activity date and a campaign activity time, as described below.

In one embodiment, a campaign administrator, e.g., on behalf of the campaign host, may access the campaign system 14 to create a campaign having at least one campaign activity. Each campaign activity may request one or more recruit to participate in the campaign activity. The campaign administrator may enable funding for the campaign and/or the at least one campaign activity. For example, the campaign administrator may provide a predetermined funding per recruit, a predetermined funding per campaign, and/or a predetermined funding per campaign activity. For example, the campaign administrator may establish a campaign with a first campaign activity and a second campaign activity. The campaign administrator may agree to compensate users, for example, for the first 50 recruits of the campaign at $5 per recruit; for the first 50 recruits of the first activity at $5 per recruit and the first 50 recruits of the second activity at $4 per recruit; for the first 50 recruits of the campaign at $5 per recruit and the second 50 recruits of the campaign at $3 per recruit; for all recruits until a maximum number of recruits is reached; for all recruits until a maximum compensation is reached in the campaign; for all recruits until a user has recruited a particular number of recruits; and/or the like or some combination thereof.

In one embodiment, the campaign administrator using the campaign system 14, or third-party in charge of a campaign at a particular location using the third-party system 15, may record participation of the recruit in the campaign activity. The campaign system 14 and/or the third-party system 15 may store the recorded participation in the intermediary system 12 and/or the accounting system 13. The campaign administrator, or third-party, may record recruit information that can be matched against the recruit that has signed up, or may record the recruit ID, or the like.

In one embodiment, when the recruit has completed a campaign activity, the recruit is presented with a participation code. The recruit may present the participation code to the user, who upon entering the participation code into the user system 16, is credited for the recruit's participation in the campaign activity.

In another embodiment, when the recruit has completed a campaign activity, the user is credited for the recruit's participation in the campaign activity without any intervention by the user. In this embodiment, the intermediary system 12 and/or the accounting system 13 may query the campaign system 14 (e.g., via the API, or other web-based method) once the scheduled campaign activity has passed to determine if the recruit participated in the campaign activity, and, if the recruit did participate in the campaign activity, will credit and/or distribute funds to the user account.

In one embodiment, the user system 16 may provide one or more user action during communication with the intermediary system 12. To that end, each user action may alter one or more database within the intermediary system 12, provide one or more reports (e.g., physical reports), and/or transmit one or more communications to the intermediary system 12. Exemplary user actions may include, but are not limited to associate recruits with a campaign activity, provide an account profile for the user, update account profile for the user, schedule the recruit for at least one campaign activity, update or edit the schedule for the recruit for the at least one campaign activity, provide an account profile for one or more recruits, view real time statistics (e.g., allocated money, funds, scheduled campaign activities, etc.). Additionally, the user system 16 may be prompted to register one or more user account with the intermediary system 12.

In one embodiment, the campaign system 14 may provide one or more campaign actions during communication with the intermediary system 12. To that end, each campaign action may alter one or more database within the intermediary system 12, provide one or more reports (e.g., physical reports), and/or transmit one or more communications to the intermediary system 12. Exemplary campaign actions may include, but are not limited to create and/or edit a campaign; add or remove campaign activities; add, edit, or remove campaign activity properties such as campaign activity funds; create, add, or edit campaign account profile; generate a billing request; view real time statistics (e.g., allocated money, campaign activity schedules); provide a timeframe for the campaign; provide a requested number of recruits during the timeframe; provide a campaign activity fund limit; provide campaign activity availability; and provide other information regarding the campaign; and/or the like. Additionally, the campaign system 14 may be prompted to register one or more campaign accounts with the intermediary system 12 and set up the campaign account (e.g., provide organization name, address, campaign plea, and/or the like).

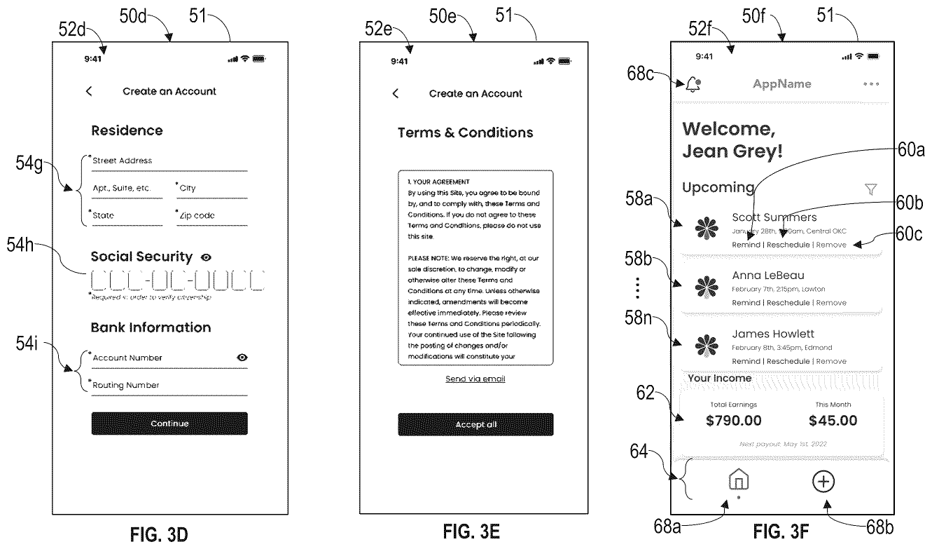

FIGS. 3A-5 illustrate an exemplary embodiment of a recruitment software application 36 for the recruitment platform 10 for use on the user system 16. The recruitment software application 36 may be downloaded onto the one or more user system 16.

Referring now to FIGS. 3A-3F in combination, shown therein are exemplary screenshots 50a-f of a user registration for the recruitment software application 36. The recruitment software application 36 may provide ability for storage and management of a user account. To that end, a collection of data associated with a particular user of the recruitment software application 36 may be stored on the intermediary system 12 in the user account. In some embodiments, at least a portion of data associated with the particular user of the recruitment software application 36 may be stored on the user system 16 associated with the particular user. Each user account may include user information, such as but not limited to, a user ID, a username, a first name, a last name, a password, one or more defined security access levels, a history of interactions (e.g., scheduled recruits, conversion rate), setting preferences, demographic data, social media contacts and/or data, and/or the like, for example.

FIG. 3A illustrates a screenshot 50a of an exemplary embodiment of a welcome screen 52a on a user interface 51 encouraging users to register or log in to the recruitment software application 36. FIG. 3B illustrates a screenshot 50b of an exemplary embodiment of a user account creation screen 52b on the user interface 51 requesting user information from the user through a series of fields 54, such as, but not limited to, a first name field 54a, a last name field 54b, a birthdate field 54c, an email address field 54d, and a password field 54e, encouraging users to register or log in to the recruitment software application 36. Once the user has input user information into the fields 54, the user may select an input 56 to continue with account creation.

In some embodiments, the user is then presented with a phone number field 54f as shown in a screenshot 50c of a phone number screen 52c on the user interface 51 of FIG. 3C, to receive the user's phone number. The recruitment software application 36 may then verify the user's phone number, e.g., by transmitting a time-sensitive code to the user's phone number that the user must then enter into a verification code field displayed on the user system 16. Once the phone number is verified, the user may be prompted with another registration screen 52d on the user interface 51, shown in screenshot 50d in FIG. 3D, including address fields 54g, social security number field 54h, and banking information fields 54i.

The user may be presented with a terms and conditions, privacy, or other policy, information screen 52e on the user interface 51 where the user may indicate that the user understands the terms and conditions, privacy, or other policy information, as shown in screenshot 50e in FIG. 3E.

In some embodiments, during the log-in and/or registration process, the user may be given an option to link the user account with one or more social media platforms (e.g., Facebook, Linked-In, Twitter, TikTok, etc.). By linking the social media platform, the intermediary system 12 may be able to determine additional information associated with the user including, but not limited to, age, demographics, schooling, location, and/or the like.

Referring to FIG. 3F, shown therein is an exemplary embodiment of screenshot 50f of a user dashboard 52f on the user interface 51. Upon completion of user signup, the user may be assigned a user ID to uniquely identify the user or the user account. The user may be directed to the dashboard after the user has created the user account and/or after the user logs into the recruitment software application 36. In one embodiment, the user dashboard includes one or more alert 58a-n indicative of a recruit status. For example, the alert may include a recruit name, scheduled campaign activity information, and an option 60a to remind the recruit of the campaign activity, an option 60b to reschedule the recruit for the campaign activity, and/or an option 60c to remove the recruit from the campaign activity. Additionally, the screenshot 50f shows an income status 62 indicating total income received by the user and income received by the user within the current month.

The user dashboard may provide a user interface that organizes and presents information in a simplified format for the user. The user dashboard may integrate information from multiple components of the recruitment software platform in a unified display for the user. For example, the user dashboard may provide one or more current metrics and/or performance indicators. Generally, the user dashboard may obtain information from the intermediary system 12, the user system 16, the third-party system 15, and/or the campaign system 14 and present it to the user. In some embodiments, the intermediary system 12 may receive information and/or data from the campaign system 14, the third-party system 15, and/or the user system 16, analyze the information and/or data and present the analyzed data on the user dashboard.

In one embodiment, when the user selects option 60a to remind the recruit of the scheduled campaign activity, the recruitment software application 36 may automatically fill out an email or text message addressed to the recruit with the scheduled campaign activity information including, for example, the campaign activity, the campaign activity ID, the campaign activity date, time, and location, and additional instructions (e.g., instructions for how to access the campaign activity location such as “ring the doorbell and ask for John Smith”, or instructions for checking in, such as “Please arrive 10 minutes early and drink plenty of water today!”).

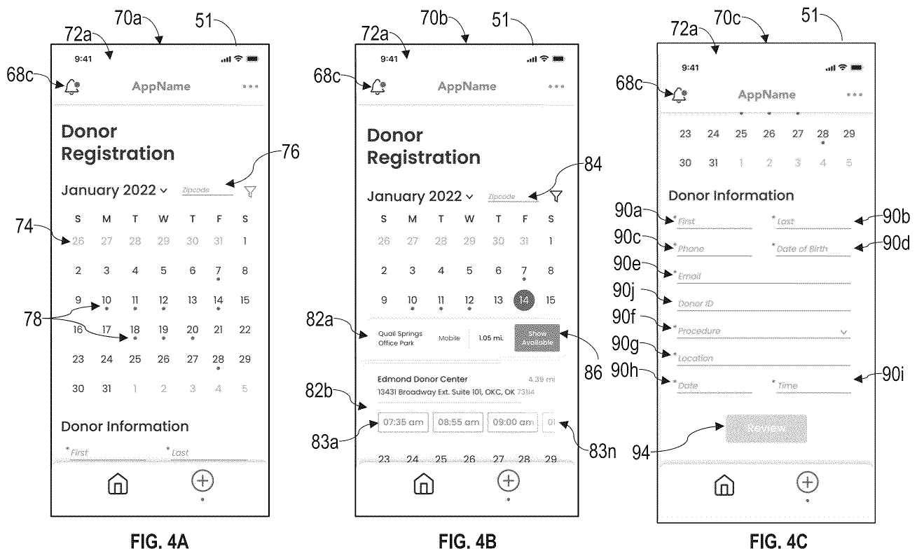

In one embodiment, when the user selects option 60b to reschedule the recruit for the campaign activity, the recruitment software application 36 may proceed to screenshot 70c (detailed below) and automatically complete the current campaign activity information into inputs 90a-n, as described below in more detail in relation to FIG. 4C.

In one embodiment, the user home screen may further include a menu 64 having at least a home input 68a and a schedule recruit input 68b. Selection of the home input 68a from the menu 64 will direct the user to the home screen and/or refresh the home screen. Selection of the schedule recruit input 68b will direct the user to a recruit scheduling screen detailed below in reference to FIGS. 4A-C. As shown, most pages of the recruitment software application 36 further include a notifications input 68c depicted as a bell icon and, when notifications are present, having a notification indicator.

Referring to FIGS. 4A-C, in combination, shown therein are exemplary screenshots 70a-c of a recruit scheduling process for the recruitment software application 36 constructed in accordance with the present disclosure. Shown in FIG. 4A, the screenshot 70a depicts a donor registration screen 72a on the user interface 51, the donor registration screen 72a having a donor registration calendar 74 for a particular zip code entered into a location input 76. The donor registration calendar 74 included campaign indicators 78 on days with an active campaign. Upon selection of a date with a campaign indicator 78, the recruitment software application 36 displays campaign details 82 as shown in screenshot 70b of FIG. 4B. Selecting the show available input 86, e.g., from campaign details 82a, will show the campaign activity availability, which may include real-time campaign activity availability. Alternatively, as shown in campaign details 82b, campaign activity availability is shown with an option for the user to select a particular available schedule slot 83a-n to schedule the recruit. In one embodiment, the campaign indicators 78 are only present after a location (e.g., zip code, city, state, address, GPS coordinates, longitude and latitude, or the like) is entered into the location input 76 (FIG. 4A). In one embodiment, when a location is entered into the location input 76, the user is prompted to provide a distance from the entered location within which to view campaign activity availability.

In one embodiment, the intermediary system 12 communicates with one or more campaign system 14 and/or one or more third-party system 15 to retrieve the one or more available schedule slot 83a-n. The third-party system 15 may be out of the control of the recruitment platform 10, that is, the third-party system 15 may be operated by an entity different from an entity operating the intermediary system 12 and/or the recruitment platform 10. In this embodiment, the intermediary system 12 may communicate with the one or more campaign system 14 to retrieve the one or more available schedule slot 83a-n, and cause the campaign system 14 to communicate with the third-party system 15 as described in more detail herein.

The screenshot 70c (of FIG. 4C) depicts a recruit information section having a plurality of recruit information inputs 90a-n to receive corresponding recruit information. The recruit information inputs 90a-n may include a first name input 90a, a last name input 90b, a phone number input 90c, a birth date input 90d, an email input 90e, a campaign activity selection input 90f, a campaign activity location input 90g, a campaign activity date input 90h, and a campaign activity time input 90i. In some embodiments, additional or fewer recruit information inputs 90a-n are included and may be dependent upon the recruit information required or suggested for a particular campaign or campaign activity. In some embodiments, the inputs 90a-n further include a recruit ID input 90j; however, in some embodiments, the recruit ID input 90j is filled out, or input by, the recruitment software application 36.

In one embodiment, the recruit information section is filled out by the user when the user schedules the recruit for a particular campaign activity. In other embodiments, the recruit may fill out the recruit information section.

In one embodiment, when the user selects the show available input 86 (depicted in FIG. 4B), the user may select an available schedule slot 83a-n, thereby causing the recruitment software application 36 to fill out, complete, or enter, the campaign activity selection input 90f, the campaign activity location input 90g, the campaign activity date input 90h, and the campaign activity time input 90i for the selected available schedule slot 83.

In one embodiment, the campaign activity selection input 90f may be a drop-down menu offering the user a number of campaign activities for which the user may schedule recruits. In one embodiment, the campaign activity selection input 90f, once selected, may be used by the recruitment software application 36 to filter available options provided for the campaign activity location input 90g, the campaign activity date input 90h, and the campaign activity time input 90i. For example, once the campaign activity selection input 90f is entered, the campaign activity location input 90g may present the user with a number of locations hosting the campaign activity selected in the campaign activity selection input 90f. Similar filtering may occur with the campaign activity date input 90h and the campaign activity time input 90i where only dates and times having available schedule slots 83 for the selected campaign activity at the selected campaign activity location are shown as options in the campaign activity date input 90h and the campaign activity time input 90i, respectively.

In one embodiment, the campaign activity location input 90g may be sorted or filtered based on a location information of the user system 16, e.g., as obtained from location services, GPS location, and/or WIFI information of the user system 16. For example, the campaign activity location input 90g may provide the nearest location to the user system 16 hosting the selected campaign activity more prominently than the furthest location to the user system 16 hosting the selected campaign activity. Sorting in this manner is additionally advantageous when the user meets the recruit in-person as the recruit is likely to be able to attend a campaign activity relatively near the in-person meeting. In one embodiment, the campaign activity location input 90g may be sorted or filtered based on the location information of the recruit as provided in the location input 76 (FIG. 4A). For example, the campaign activity location input 90g may be sorted or filtered based on a ZIP code of the recruit.

Once the user has completed the required recruit information section shown in FIG. 4C, the user may select the review input 94 thereby allowing the user and/or recruit to review the information input into one or more of the inputs 90a-n prior to confirming the recruit information and scheduling the recruit for a particular campaign activity. In some embodiments, when the review input 94 is selected, a notification is sent to the phone number provided in phone number input 90c or to the email address provided in the email input 90e to which the recruit may accept the scheduled campaign activity. In some embodiments, when the review input 94 is selected, a notification having campaign activity information and a recruit confirmation request is sent to the phone number provided in phone number input 90c and/or the email address provided in email input 90e. The recruit may respond to the recruit confirmation request to confirm that the recruit has been recruited by the user and/or to confirm registration of the recruit for the scheduled particular campaign activity. In one embodiment, the recruit confirmation request is a recruit confirmation link that, when selected or clicked-on by the recruit, causes the user system 16 of the recruit to transmit a recruit confirmation to the intermediary system 12, e.g., via the network 22, thereby confirming registration of the recruit. In one embodiment, the notification is an SMS message, an MMS message, an email, a phone notification, or an instant messaging service message, such as iMessage (Apple, Inc., Cupertino, California), and/or the like.

In one embodiment, once the registration for the recruit has been confirmed, the intermediary system 12 may communicate the recruit confirmation to the one or more third-party system 15 to store the registration for the user's appointment into the third party system 15. The intermediary system 12 may send one or more recruit information to the third-party system 15, e.g., via the campaign system 14, where the one or more recruit information sent may be determined by a required information identified by the third-party system 15, e.g., as stored in the partner database in the memory 40. For example, the partner database may store data indicative of recruit information required by the third-party system 15 such as first name, last name, date of birth, scheduled time, scheduled date, and scheduled location. The required recruit information may be needed by the third-party system 15 in order to process the recruit confirmation and finalize scheduling of the recruit into the third-party system 15. In this case, the intermediary system 12 may send only the required information to the third-party system 15.

In some embodiments, the third party system 15 maintains a master schedule or calendar of the filled and available schedule slots. But, the processor 38 of the campaign system 14 may be receiving requests to schedule multiple recruits simultaneously due to multiple user systems 16 scheduling the recruits simultaneously. The memory 40 of the campaign system 14 may store a time series of scheduling requests. To prevent multiple recruits from being inadvertently scheduled for a same schedule slot, when the processor 38 requests available scheduling slots from the third party system 15, the processor 38 also accesses the time series of scheduling requests stored in the memory 40. Then, the processor 38 validates the available scheduling slots received from the third party system 15 with the scheduling requests stored in the memory 38 and removes any of the available scheduling slots received from the third party system 15 if such available scheduling slot is subject to a scheduling request indicating that such available scheduling slot may actually be filled.

For example, in one embodiment, the processor 38 of campaign system 14 may analyze and/or intercept one or more communication between the campaign host, campaign administrator, or other user, and the one or more third-party system 15 where the one or more communication is operable to cause the user system 16 to display a first interface having one or more available schedule slot. For example, if the third-party system 15 provides a first interface showing availability of a campaign activity (e.g., one or more available schedule slot), the processor 38 of the campaign system 14 may alter the first interface to remove availability of a campaign activity corresponding to one or more scheduled campaign activity prior to the scheduled campaign activity being entered into the third-party system 15 by the campaign host or campaign administrator. In one embodiment, the campaign system 14 may further provide a second interface, e.g., over at least a portion of the first interface, wherein the user may select an input of the second interface to cause the processor 38 to insert information (such as the one or more scheduled campaign activity and/or required recruit information) displayed in the second interface into the first interface or to provide an unavailable indicator in the first interface on the one or more available schedule slot corresponding to the activity date and the activity time of the scheduled campaign activity.

In one embodiment, once the registration for a recruit has been confirmed (either by the campaign system 14 or the third-party system 15, for example), the intermediary system 12 may communicate the recruit confirmation with the one or more reconciliation circuitry of the campaign application 42 in the campaign system 14. The intermediary system 12 may send one or more recruit information to the reconciliation circuitry where the one or more recruit information sent may be determined by a required recruit information identified by the third-party system 15. In one embodiment, the required recruit information may be stored in the memory 40, such as in the partner database. An administrator may access the reconciliation circuitry and approve or deny the registration for a recruit prior to the transmission of the recruit confirmation and required recruit information to the third-party system 15. In some embodiments, the reconciliation circuitry may be notified, e.g., via an API call, a message queueing system, and/or the like, by the third-party system 15 that the registration for the recruit is ready to be reconciled.

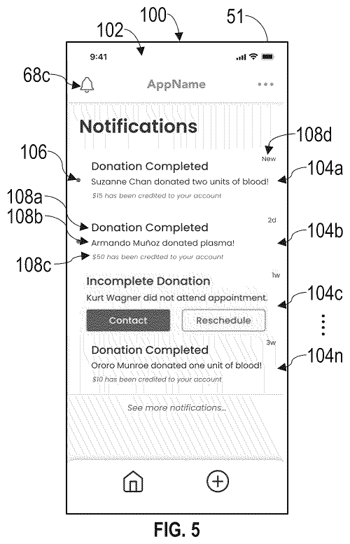

Referring now to FIG. 5, shown therein is an illustration of an exemplary embodiment of a screenshot 100 of the recruitment software application 36 showing a notifications screen 102 in the user interface 51 constructed in accordance with the present invention. When the notification input 68 is available for the user to select, the user may select the notification input 68c to direct the user to a notification screen as depicted in the screenshot 100 shown in FIG. 5. The notification screen depicts one or more notification 104a-n. Each notification 104 may include a read indicator 106 indicative of whether the user has read a particular notification 104, e.g., whether the user has selected the particular notification 104, whether the user has marked the particular notification as read, or whether the user has been presented with the particular notification, for example.

Each notification 104a-n may include a plurality of notification information 108a-n including, for example, a campaign activity status 108a, a recruit status 108b, a funding status 108c, and a notification time indicator 108d. In other embodiments, additional or fewer notification information 108 may be included with each notification 104, such as a date completed, time completed, or the like, for example. Other notification information may include whether the recruit completed the scheduled campaign activity at the scheduled date and time or whether the recruit attempted the campaign activity but was unable to complete the campaign activity, for example.

In one embodiment, when the notification 104 is indicative of a recruit that missed the scheduled campaign activity, such as shown in notification 104c, the user may be presented with an option to send a contact to the recruit, e.g., to send a communication to the recruit, and/or to reschedule the recruit for a different date or time to complete the campaign activity.

In some embodiments, the user may receive a notification, such as a notification 104, indicating that a bonus has been issued for recruits scheduled for a particular campaign activity at a particular location. For example, a campaign administrator may issue a location bonus for all recruits that donate blood at a particular blood donation location. A campaign administrator may also issue a blood-type bonus, for example, for all recruits of a particular blood type that donate blood at a particular blood donation location and may issue a blood-property bonus for all recruits with a particular blood property, e.g., rh-negative, that donate blood at a particular blood donation location. In these examples, the user may receive a base rate for the recruit donating blood, the location bonus, the blood-type bonus, and the blood-property bonus if the recruit meets all of the requirements for each bonus.

In another embodiment, the user may receive a bonus for any recruit that completes a campaign activity wherein the recruit is a lapsed recruit. A lapsed recruit may be any recruit that has previously completed a campaign activity but has not completed a campaign activity again within a particular period of time. For example, if a particular recruit has donated blood (i.e., completed a campaign activity) in the past, but has not donated blood within the last year, for example, the recruit may be considered a lapsed recruit. The user, having scheduled a lapsed recruit where the lapsed recruit completes the scheduled campaign activity, may receive a bonus for scheduling a lapsed recruit.

In one embodiment, the user, having scheduled a particular recruit, may receive a first campaign activity fund when the recruit completes the campaign activity and may receive a second campaign activity fund each time the recruit completes a campaign activity within a predetermined period of time and while the recruit is associated with the user. For example, the user may receive a first campaign activity fund the first time the recruit donates blood (i.e., completes the campaign activity) and may receive a second campaign activity fund the second time the recruit donates blood, even if the user did not schedule the recruit for the second campaign activity.

In some embodiments, the second campaign activity fund is lesser than the first campaign activity fund. In some embodiments, if the first campaign activity fund included a particular bonus (e.g., a blood-type bonus) the second campaign activity fund will also the particular bonus (e.g., the blood-type bonus). Whether a bonus will continue for each activity the recruit completes within the predetermined period of time may be determined by the campaign administrator when the campaign administer establishes the campaign and/or establishes the bonus structure for the campaign activity. Likewise, the predetermined period of time may be determined by the campaign administrator when the campaign administer establishes the campaign and/or establishes the bonus structure for the campaign activity.

In one embodiment, when a first user schedules a recruit, the recruit is associated with the first user for the predetermined period of time. However, if a second user schedules the recruit within the predetermined period of time, the second user may receive a campaign activity fund and/or a bonus for scheduling the recruit, while the first user no longer receives any further campaign activity fund or bonus for the recruit.

The recruitment platform systems and methods disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

What is claimed is:

1. A system, comprising:
a processor; and
a memory comprising one or more non-transitory processor-readable medium storing processor-executable instructions that when executed by the processor cause the processor to:
display, on a user system of a user, a user interface to enable a campaign activity associated with a campaign, the user interface having one or more campaign activity property, and one or more input fields corresponding to recruit information, the recruit information corresponding to a particular recruit, the campaign activity being a tissue donation;
receive, from the user system, a user ID, the recruit information from the one or more input fields, and an activity indicator identifying a particular one of the one or more campaign activity;
schedule the particular one of the one or more campaign activity with the recruit information;
receive, from a campaign system, a recruit identifier and a recruit status, the recruit identifier identifying the particular recruit and the recruit status indicative of whether the recruit completed the campaign activity; and
allocate, to the user of the user system, at least one of a donation or a monetary value based upon the one or more campaign activity property when the recruit status is indicative of the particular recruit successfully completing the campaign activity.

2. The system of claim 1, wherein the one or more campaign activity property is one or more of a campaign activity date, a campaign activity time, a campaign activity location, a campaign activity ID, and a campaign activity fund.

3. The system of claim 2, wherein the activity indicator is the campaign activity ID or a combination of the campaign activity date, the campaign activity time, and the campaign activity location.

4. The system of claim 1, wherein the recruit status is further indicative of whether the recruit did not attend the campaign activity; attended the campaign activity but did not complete the campaign activity; or attended the campaign activity and did complete the campaign activity.

5. The system of claim 1, wherein the campaign is a blood donation campaign.

6. The system of claim 1, wherein the campaign activity is a blood donation activity.

7. The system of claim 1, wherein the campaign activity is a plasma donation activity.

8. The system of claim 2, wherein the memory stores processor-executable instructions that further cause the processor to:
allocate, to a user account of the user, a monetary value based at least in part on the campaign activity fund; and
transmit, to the user, the monetary value.

9. A method, comprising:
receiving from a user system associated with a user, a user ID, and recruit information identifying a recruit different from the user;
scheduling an activity date and an activity time for a tissue donation campaign activity to be completed by the recruit;
updating a recruit status to indicate the recruit successfully completing the tissue donation campaign activity; and
allocating, to the user of the user system, at least one of a donation or a monetary value based upon the recruit status indicating the recruit successfully completing the campaign activity.

10. The method of claim 9, wherein the recruit status is further indicative of whether the recruit did not attend the campaign activity; attended the campaign activity but did not complete the campaign activity; or attended the campaign activity and did complete the campaign activity.

11. The method of claim 9, wherein the campaign activity is a blood donation activity.

12. The method of claim 9, wherein the campaign activity is a plasma donation activity.

13. A system, comprising:
a processor; and
a memory comprising one or more non-transitory processor-readable medium storing processor-executable instructions that when executed by the processor cause the processor to:
provide, to a user system of a user, a user interface to enable one or more campaign activity associated with a campaign, each campaign activity associated with a tissue donation campaign and the user interface having one or more campaign activity property, and one or more input fields corresponding to recruit information, the recruit information corresponding to a particular recruit;
receive, from the user system, a user ID, the recruit information from the one or more input fields, and an activity indicator identifying a particular campaign activity of the one or more campaign activity;
transmit, to a campaign system, the recruit information and an activity date and an activity time corresponding to the particular campaign activity;
receive, from the campaign system, a recruit identifier and a recruit status, the recruit identifier identifying the particular recruit and the recruit status indicative of whether the recruit completed the particular campaign activity; and
allocate, to the user of the user system, at least one of a donation and a monetary value, the at least one of a donation and a monetary value based upon the one or more campaign activity property when the recruit status is indicative of the recruit successfully completing the particular campaign activity.

14. The system of claim 13, wherein the memory further stores instructions that cause the processor to:
analyze one or more communication between a third-party system and the user, the one or more communication operable to cause the user system of the user to display a first interface having one or more available schedule slot; and
provide one or more second interface showing the recruit information, the activity date, and the activity time corresponding to the particular campaign activity.

15. The system of claim 14, wherein the memory further stores instructions that cause the processor to:
modify the first interface provided by the third-party system to display an unavailable indicator on at least one of the one or more available schedule slot corresponding to the activity date and the activity time of the particular campaign activity.

16. The system of claim 14, wherein the memory further stores instructions that cause the processor to:
display, on the second interface, an input operable to, upon selection by the user, cause the processor to insert one or more of the recruit information, the activity date, and the activity time into the first interface.

17. The system of claim 14, wherein the one or more second interface is provided over the first interface.

18. The system of claim 13, wherein the campaign is a blood donation campaign.

19. The system of claim 13, wherein at least one of the one or more campaign activity is a blood donation activity.

20. The system of claim 13, wherein at least one of the one or more campaign activity is a plasma donation activity.
